(19) United States
(12) Patent Application Publication
(10) Pub. No.: US 2010/0028872 A1
(43) Pub. Date: Feb. 4, 2010

(54) Methods for the Detection of Cancer

(76) Inventor: Paul Cairns, Philadelphia, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/279,127

(22) PCT Filed: Feb. 13, 2007
(86) PCT No.: PCT/US6007/062060

§ 371 Date: Apr. 7, 2009

Related U.S. Application Data
(60) Provisional application No. 60/772,699, filed on Feb. 13, 2006.

(51) Int. Cl. C12Q 1/68

(52) U.S. Cl. 435/6

(57) ABSTRACT

Methods, compositions, and kits for the detection of cancer, particularly renal cancer, are provided.

This application claims priority under 35 U.S.C. §119(e) to U.S. Provisional Patent Application No. 60/772,699, filed on Jan. 13, 2006. The foregoing application is incorporated by reference herein.

Pursuant to 35 U.S.C. §202(c), it is acknowledged that the U.S. Government has certain rights in the invention described herein, which was made in part with funds from the National Institutes of Health, Grant Number UO1 1619601.

### FIELD OF THE INVENTION

This invention relates to the fields of oncology and molecular biology. More specifically, the present invention provides methods for detecting the presence of cancer, particularly renal cancer, based on the promoter methylation pattern of a panel of genes.

### BACKGROUND OF THE INVENTION

Several publications and patent documents are cited throughout the specification in order to describe the state of the art to which this invention pertains. Each of these citations is incorporated herein by reference as though set forth in full.

There will be more than 30,000 new cases of renal cell cancer (RCC) in 2006 and the incidence of this disease has increased by 60% over the last two decades (Jemal et al. (2005) CA Cancer J. Clin., 55:10-30). RCCs are heterogeneous in histology, genetics and clinical behavior. Clear-cell (75-80%) and papillary (10-15%) carcinoma are the two most frequent subtypes of RCC (Kovacs et al. (1997) J. Pathol., 183:131-3; Storkel et al. (1997) Cancer, 80:987-9; Zambrano et al. (1999) J. Urol., 162:1246-58).

Tumorigenesis is a multi-step process that results from the accumulation and interplay of genetic and epigenetic mutations. The epigenetic alteration of aberrant DNA methylation of CpG islands in the promoter region of genes is well established as a common mechanism for the silencing of a tumor suppressor gene (TSG) in cancer cells (Baylin et al. (1998) Adv. Cancer Res., 72:141-96; Jones et al. (1999) Nature Genet., 21:163-67). Several TSG have been identified as hypermethylated with associated loss of expression in renal cancer by a candidate approach. The tumor suppressor genes VHL and p16^{INK4a} are inactivated by promoter hypermethylation in up to 20% of clear-cell and 10% of all RCC, respectively (Herman et al. (1994) Proc. Natl. Acad. Sci., 91:9700-4; Herman et al. (1995) Cancer Res., 55:4525-30). However, to date, few genes have been found to be frequently hypermethylated in RCC. For example, the RASSF1A gene is hypermethylated in 27% to 56% and the Timp-3 gene is hypermethylated in 58% to 78% of primary RCC (Morissey et al. (2001) Cancer Res., 61:7277-81; Yoon et al. (2001) Int. J. Cancer, 94:212-7; Dulaimi et al. (2004) Clin. Cancer Res., 10:3972-9; Bachman et al. (1999) Cancer Res., 59:798-802). By definition, a candidate gene approach has resulted in the examination of a limited number of genes for epigenetic alteration (Dulaimi et al. (2004) Clin. Cancer Res., 10:3972-9). Accordingly, many other tumor suppressor and cancer genes important in renal tumorigenesis likely remain to be identified.

### SUMMARY OF THE INVENTION

In accordance with the present invention, a method for the detection of cancer, particularly renal cancer, is provided. In general, the instant invention provides methods for the detection of cancer by detecting the methylation state of at least one tumor suppressor gene promoter region. In one embodiment, the tumor suppressor gene promoter region is selected from the genes provided in FIGS. 4-7. In another embodiment, the tumor suppressor gene promoter region is selected from the group consisting of the IGFBP1, IGFBP3, COL1A1, GDF15, and PLAU. In a particular embodiment, the tumor suppressor promoter region is the promoter region of IGFBP1. An exemplary method entails providing a biological sample obtained from a patient and performing methylation specific PCR (MSP) on the nucleic acid molecules of the biological sample. The hypermethylation of the nucleic acids molecules obtained from the patient, in comparison to that from a normal subject, is indicative of renal cancer.

According to one embodiment of the instant invention, the nucleic acid molecules comprise one or more tumor suppressor gene promoter regions. In a particular embodiment, at least one nucleic acid molecule of the biological sample is isolated prior to MSP. In yet another embodiment, a biological sample obtained from a normal subject can be analyzed along side the biological sample obtained from a patient. In still another embodiment, the MSP is performed on at least one tumor suppressor gene promoter region. In a preferred embodiment of the instant invention, the at least one tumor suppressor gene promoter region comprises at least one promoter region of a tumor suppressor gene selected from the group consisting of the IGFBP1, IGFBP3, COL1A1, GDF15, and PLAU. In another embodiment, the at least one tumor suppressor gene promoter region comprises at least the IGFBP1, IGFBP3, and COL1A1 promoter regions. In another embodiment, the at least one tumor suppressor gene promoter region comprises at least the IGFBP1 promoter region.

According to another aspect of the invention, kits for performing the methods described above are provided. Exemplary kits of the instant invention comprise at least one set of primers specific for performing methylation specific PCR of the promoter region of at least one of the genes provided in FIGS. 4-7, more particularly, at least one of the genes is selected from the group consisting of IGFBP1, IGFBP3, COL1A1, GDF15, and PLAU. In a particular embodiment, the kit comprises a set of primers specific for the promoter region of IGFBP1. The kit may further comprise at least one hypermethylated nucleic acid molecule for use as a positive control or at least one agent (e.g., Sss I methylase) to methylate a nucleic acid molecule as a positive control. The kits may further comprise at least one unmethylated nucleic acid molecule for use as a negative control. The kits may also comprise nucleic acid molecules isolated from a normal subject wherein the nucleic acid molecules comprise the promoter region of at least one of the genes selected from the group consisting of IGFBP1, IGFBP3, COL1A1, GDF15, and PLAU. The kits of the instant invention may also comprise at least one of the following: reagents suitable for performing non-denaturing gel electrophoresis, reagents for performing MSP (for example, without limitation, sodium bisulfite, polymerase, dNTPs, buffers, and tubes), and instruction material.

### BRIEF DESCRIPTION OF THE DRAWINGS

FIGS. 1A and 1B consist of images of gels showing the DNA product of reverse transcription and PCR amplification from untreated (U) and drug treated (T) RCC cell lines.

FIGS. 2A-2H provide graphical representation of the sequencing of gene promoters in normal and tumor DNAs after bisulfite modification. Each pair shows the DNA sequence from a normal tissue and from one of the cell lines described herein. Unmethylated cytosines (C) are converted to uracil (T). The presence of C preceding a G in the sites indicated by arrows demonstrates that these cytosines were methylated in the cell line DNA while the presence of T instead of C in the same positions in the normal DNA demonstrates that these C were unmethylated in the normal tissue DNA. SEQ ID NOs: 65 and 66 (FIG. 2A), SEQ ID NOs: 67 and 68 (FIG. 2B), SEQ ID NOs: 69 and 70 (FIG. 2C), SEQ ID NOs: 71 and 72 (FIG. 2D), SEQ ID NOs: 73 and 74 (FIG. 2E), SEQ ID NO: 75 (FIG. 2F), SEQ ID NOs: 76-78 (FIG. 20, top to bottom), and SEQ ID NOs: 79-81 (FIG. 2H, top to bottom) are provided.

FIG. 3A consists of images of gels showing the presence of the PCR product from methylation specific PCR. M indicates methylated and U indicates unmethylated. Tumor cell lines 786-0 for IGFBP1 and ACHN for IGFBP3 and COL1A1 were used as positive controls (+ve) and normal renal cell DNA was used as a negative control (−ve). FIG. 3B consists of an image of a gel showing the presence of the PCR product from methylation specific PCR on the 4 RCC cell lines. M indicates methylated and U indicates unmethylated. Normal renal cell DNA was used as a negative control (−ve) and cell line MDA231 was used as a positive control (+ve).

FIGS. 4A-4C provides a list of the genes upregulated in the 786-0 cell line.

FIGS. 5A-5B provides a list of the genes upregulated in the HRC51 cell line.

FIGS. 6A-6D provides a list of the genes upregulated in the HRC59 cell line.

FIGS. 7A-7B provides a list of the genes upregulated in the ACHN cell line.

### DETAILED DESCRIPTION OF THE INVENTION

While renal cancer is exemplified throughout this application, the methods of the instant invention can be used for the detection of any cancer. In a particular embodiment, the cancer may be selected from the group consisting of, without limitation, cancers of the prostate, colorectum, pancreas, cervix, stomach, endometrium, brain, liver, bladder, ovary, testis, head, neck, skin, melanoma, basal carcinoma, mesothelial lining, white blood cells, lymphoma, leukemia, esophagus, breast, muscle, connective tissue, lung, small-cell lung carcinoma, non-small-cell carcinoma, adrenal gland, thyroid, kidney, or bone; glioblastoma, mesothelioma, renal cell carcinoma, gastric carcinoma, sarcoma, choriocarcinoma, cutaneous basocellular carcinoma, and testicular seminoma. In a particular embodiment, the cancer is selected from the group consisting of renal, bladder, ovarian, and colorectal. In a particular embodiment, the cancer is renal cancer.

Kidney cancer confined by the renal capsule can be surgically cured in the majority of cases whereas the prognosis for patients with advanced disease at presentation remains poor. Novel strategies for early detection of renal cancer, as with all cancers, are therefore needed. Promoter hypermethylation is a common mechanism for tumor suppressor inactivation in human cancer. The instant invention demonstrates that the hypermethylation of at least one of the tumor suppressor genes IGFBP1 (GenBank Accession No. NM_{—}000596), IGFBP3 (GenBank Accession No. NM_{—}000598), COL1A1 (GenBank Accession No. NM_{—}000088), GDF15 (GenBank Accession No. NM_{—}004864), and PLAU (GenBank Accession No. NM_{—}002658) is an early indicator of renal cancer.

A global approach to the identification of epigenetically silenced genes in renal tumor cells may provide methylation signatures for early detection and for prognostic stratification, identify novel targets for therapy, and lead to further elucidation of the biology of this disease. Epigenetic silencing of a gene can be reversed by drugs such as 5Aza-2deoxycytidine (5Aza-dC), thereby resulting in re-expression. 5Aza-dC acts by incorporation into the new strand during DNA replication where it forms a covalent complex with the methyltransferase active sites, thereby depleting methyltransferase activity and generalized demethylation (Baylin et al. (1998) Adv. Cancer Res., 72:141-96). Trichostatin A (TSA) is a histone deacetylase inhibitor agent that can reverse the formation of transcriptionally repressive chromatin structure by facilitating an accumulation of acetylated histones (Marks et al. (2004) Adv. Cancer Res., 91:137-68). These two drugs have been reported to act in synergy for reactivation of epigentically silenced genes (Cameron et al. (1999) Nat. Genet., 21:103-7).

Until recently, global analyses of methylation in cancer cells were largely restricted to array or gel-based comparisons of CpG islands between normal and tumor cell DNA. Previous global strategies to identify methylated genes in cancer tended to use arrayed CpG island fragments identified through restriction enzyme recognition sequences (Costello et al. (2000) Nat. Genet., 24:132-8; Wei et al. (2002) Clin. Cancer Res., 8:2246-52). One issue with such an approach is that many CpG islands are located outside promoter regions and methylation of such islands does not have a functional effect upon transcription (Suzuki et al. (2002) Nat. Genet., 31:141-9). A microarray-based screen has the advantage of a more global analysis and, coupled with a reactivation strategy, has the further advantage that it should preferentially identify re-expression of epigenetically silenced genes over methylated CpG islands that do not influence transcription. The potential of this approach has been highlighted in bladder, colorectal, esophageal, and other cancers (Liang et al. (2001) Cancer Res., 62:961-6; Suzuki et al. (2002) Nat. Genet., 31:141-9; Yamashita et al. (2002) Cancer Cell, 2:485-95; Sato et al. (2003) Cancer Res., 63:3735-42; Tokumaru et al. (2004) Cancer Res., 64:5982-7; Lodygin et al. (2005) Cancer Res., 65:4218-27). The SPINT2 gene was recently identified as methylated in 40% of papillary and 30% of clear cell renal tumors after demethylation treatment of RCC lines (Morris et al. (2005) Cancer Res., 4598-606). These epigenetic reactivation profiles may in part also be array-type dependent as well as sensitive to different 5Aza-dC doses and treatment times.

In the present study, the global reactivation of epigenetically silenced genes in renal cancer was examined by analysis of a 14,802 gene expression array with RNA from 4 RCC lines after treatment with 5Aza-dC and TSA. Through validation, the specificity of the screen was demonstrated for epigenetically silenced genes and 3 genes unmethylated in normal cells but frequently hypermethylated in primary RCC were identified.

Aberrant promoter hypermethylation is a common mechanism for inactivation of tumor suppressor genes (TSG) in cancer cells. To generate a global profile of genes silenced by hypermethylation in renal cell cancer (RCC), an expression microarray-based analysis of genes reactivated in RCC lines after treatment with the demethylating drug 5Aza-2 deoxycytidine (5Aza-dC) and histone deacetylation inhibiting drug trichostatin A (TSA) was performed. First, the optimal drug treatment level was determined by testing for sensitive reactivation on the array of several tumor suppressor genes, well characterized as silenced by promoter hypermethylation, in the SW48 colorectal tumor cell line. The global expression pattern of a 14,802 gene oligoarray was then analyzed in drug-treated versus untreated RCC lines, 786-0, HRC51 (clear cell), ACHN and HRC59 (papillary). Between 111 and 170 genes were found to have at least 3-fold upregulation of expression after treatment in each cell line. To establish the specificity of the screen for identification of genes epigentically silenced in cancer cells, a subset of 28 genes upregulated after treatment was selected for validation. Twelve of the 28 genes had a CpG island in the promoter region as well as expression in normal renal cells. The promoter methylation status and transcription status of this subset of 12 reactivated genes were validated by semi-quantitative RT-PCR of untreated and treated cell line cDNA and by bisulfite sequencing and methylation specific PCR (MSP) of tumor cell line, primary renal tumor and normal cell DNA. Three of the 12 genes (IGFBP1, IGFBP3 and COL1A7) showed promoter methylation in tumor DNA but were unmethylated in normal cell DNA, one gene (GDF15) was methylated in normal cells but more densely methylated in tumor cells, one gene (PLAU) showed cancer cell specific methylation with some correlation to expression status, and one gene (TGM2) was unmethylated but known to be regulated by another gene (RASSF1A) methylated in renal cancer cells. Thus, it was demonstrated that upregulation of at least 6 of the 12 genes examined due to epigenetic reactivation supporting the specificity of our screen. The IGFBP1, IGFBP3 and COL1A1 gene promoter regions were found to be frequently methylated in primary renal cell tumors and are candidate TSG.

### I. DEFINITIONS

The following definitions are provided to facilitate an understanding of the present invention.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, may refer to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. Alternatively, this term may refer to a DNA that has been sufficiently separated from (e.g., substantially free of) other cellular components with which it would naturally be associated. “Isolated” is not meant to exclude artificial or synthetic mixtures with other compounds or materials, or the presence of impurities that do not interfere with the fundamental activity, and that may be present, for example, due to incomplete purification.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (see Sambrook et al. (2001) Molecular Cloning. A Laboratory Manual, Cold Spring Harbor, N.Y.:Cold Spring Harbor Laboratory Press):

Tₘ=81.5° C.+16.6 Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “gene” refers to a nucleic acid comprising an open reading frame encoding a polypeptide, including both exon and (optionally) intron sequences. The nucleic acid may also optionally include non coding sequences such as promoter or enhancer sequences. The term “intron” refers to a DNA sequence present in a given gene that is not translated into protein and is generally found between exons.

The term “promoter” or “promoter region” generally refers to the transcriptional regulatory regions of a gene. The “promoter region” may be found at the 5′ or 3′ side of the coding region, or within the coding region, or within introns. Typically, the “promoter region” is a nucleic acid sequence which is usually found upstream (5′) to a coding sequence and which directs transcription of the nucleic acid sequence into mRNA. The “promoter region” typically provides a recognition site for RNA polymerase and the other factors necessary for proper initiation of transcription.

The phrase “methylation specific polymerase chain reaction” refers to a method to determine the methylation status of nucleic acid molecules. The nucleic acid molecules may comprise CpG islands. This approach allows the determination of methylation patterns from very small samples of DNA, including those obtained from paraffin-embedded samples, and can be used in the study of abnormally methylated CpG islands in neoplasia. Methylation-specific PCR is described, for example, in U.S. Pat. Nos. 5,786,146; 6,200,756; 6,017,704; and 6,265,171 and U.S. Patent Application Publication No. 2004/0038245.

The phrase “tumor suppressor genes” refers to a class of genes involved in different aspects of normal control of cellular growth and division, the inactivation of which is often associated with oncogenesis. The phrase “tumor suppressor genes” may also refer to those genes whose expression within a tumor cell suppresses the ability of such cells to grow spontaneously and form an abnormal mass, i.e., the expression of which is capable of suppressing the neoplastic phenotype and/or inducing apoptosis.

As used herein, the term “biological sample” refers to a subset of the tissues (e.g., renal tissue) of a biological organism, its cells, or component parts (e.g. body fluids such as, without limitation, blood, serum, plasma, and peritoneal fluid). In a particular embodiment, the biological sample is selected from the group consisting of urine, kidney tissue, and tumor tissue.

As used herein, an “instructional material” includes a publication, a recording, a diagram, or any other medium of expression which can be used to communicate the usefulness of the compositions described herein for performing a method of the invention. The instructional material of the kits presently claimed can, for example, be affixed to a container which contains a kit of the invention to be shipped together with a container which contains the kit. Alternatively, the instructional material can be shipped separately from the container with the intention that the instructional material and kit be used cooperatively by the recipient.

### II. KITS

Kits are provided for practicing the methods of the instant invention. The kits may comprise primers specific for performing methylation specific PCR of the promoter region of at least one of the tumor suppressor genes selected from the group consisting of IGFBP1, IGFBP3, COL1A1, GDF15, and PLAU. In a particular embodiment, the kits may comprise primers specific for performing methylation specific PCR of the promoter region of at least one of the tumor suppressor genes selected from the group consisting of IGFBP1, IGFBP3, and COL1A1. In yet another embodiment, the kits may comprise primers specific for performing methylation specific PCR of the promoter region of IGFBP1.

The kits of the instant invention may also further comprise at least one hypermethylated nucleic acid molecule for use as a positive control and/or or at least one agent (e.g., Sss I methylase) to methylate a nucleic acid molecule as a positive control. The kit may also further comprise at least one further item selected from the group consisting of at least one unmethylated nucleic acid molecule for use as a negative control, at least one reagent suitable for performing methylation specific PCR (for example, without limitation, sodium bisulfite, polymerase, dNTPs, buffers, and tubes), at least one reagent suitable for performing non-denaturing gel electrophoresis (see, e.g., Ausubel et al. (2005) Current Protocols in Molecular Biology, John Wiley and Sons, New York), and instruction material. The kit may also further comprise at least one sample container.

The Example set forth below is provided to better illustrate certain embodiments of the invention. It is not intended to limit the invention in any way.

### Example

### Methods

### Cell Lines and Tissue Specimens

The clear cell renal tumor line 786-0 and the papillary renal tumor cell line ACHN were obtained from the American Type Culture Collection (ATCC). The HRC51 cell line was established from an organ-confined primary clear cell renal tumor (T1b, grade I) and the HRC59 cell line from a localized papillary renal tumor (T3a, grade III) at Fox Chase Cancer Center (FCCC). 786-0 was grown in RPMI, ACHN in MEM and HRC51 and 59 in Type IIA medium supplemented with 10% Fetal Calf Serum. Primary renal tumors and normal kidney tissue were microdissected with the assistance of a pathologist as described in Battagli et al. and DNA extracted using conventional techniques of digestion with proteinase K (Invitrogen, Carlsbad, Calif.) followed by phenol/chloroform extraction (Battagli et al. (2003) Cancer Res., 63:8695-9; Sambrook et al. (2001) Molecular Cloning. A Laboratory Manual, Cold Spring Harbor, N.Y.:Cold Spring Harbor Laboratory Press).

5Aza-2deoxycytidine and TSA Treatment

5Aza-dC (Sigma, St. Louis, Mo.) was dissolved in phosphate buffered saline (PBS) as a 5 mM stock solution, and stored in aliquots at −80° C. TSA (Wako, Richmond, Va.) was dissolved in absolute ethanol as a 330 μM stock solution, and stored at −20° C. The 4 renal cell lines were split to low density and exposed to 5Aza-dC at a final concentration of 5 μM, again 24 hours after the beginning of treatment and, if necessary, further treatment until the cells had undergone at least 2 doublings. The cells were also treated with TSA at a final concentration of 500 nM during the 24 hours prior to harvest for RNA extraction. Untreated (mock) cells were cultured over an identical period of time with an equivalent volume of PBS and, for the final 24 hours, with an equivalent volume of EtOH.

### Oligonucleotide Array Hybridization

Total RNA was isolated from drug-treated and untreated cultured cells using TRIZOL reagent (Invitrogen, Carlsbad, Calif.) and purified with the RNAeasy Mini Kit (Quiagen, Valencia, Calif.) combined with DNAase treatment. The RNA quality was confirmed by the ratio of 28S and 18S ribosomal RNA after agarose gel electrophoresis. Twenty μg of total RNA was reverse transcribed using oligo (dT)24 primer and Superscript II reverse transcriptase (Invitrogen, Carlsbad, Calif.) for 1.5 hours at 42° C.

For each cell line, both treated and untreated cDNA was labeled with Cy3 or Cy5 (Amersham Biosciences, Piscataway, N.J.) and then hybridized to separate human 15k oligoarrays (MWG-Biotech Inc, High Point, N.C.) according to the manufacturer's recommendation for 18 hours at 42° C. The microarrays were processed and spotted in the DNA Microarray Facility at FCCC. The Gene list, Gene ID and Template files can be viewed at research.fccc.edu/facilities/microarray. In addition, microarray hybridization was performed with the opposite labels (dye flip) from each cell line. The hybridized slides were scanned using a GMS 428 Scanner (Affymetrix, Santa Clara, Calif.) to generate high-resolution images for both Cy3 and Cy5 channels. Image analysis was performed using the ImaGene software (BioDiscovery, Inc, El Segundo, Calif.).

Analysis of Expression Upregulation after Demethylating Treatment

The genes corresponding to each oligonucleotide spotted on the array were identified using an optimized segmentation algorithm. Spots of poor quality, as well as spots with signal levels indistinguishable from the background, were excluded. The image data were extracted and used for data analysis. Data were analyzed using the GeneSight software (BioDiscovery, Inc, El Segundo, Calif.) which includes background subtraction, data normalization (Lowess transformation), calculation of ratios, and statistical analysis of replicate spots and slides.

### Selection of Genes for Validation

A subset of genes was selected for validation by the following criteria. Genes were chosen that showed at least 3-fold upregulation in at least three of the four cell lines. Those genes with no evidence of expression in normal renal tissue according to the Cancer Genome Anatomy Project (CGAP) Serial Analysis Gene Expression (SAGE) database (cgap.nci.nih.gov) were excluded. The promoter region using the GeneCard website (bioinfo.weizmann.ac.il/cards/index.html) was analyzed to obtain the genomic and cDNA sequences from the upregulated genes. The CpG island most proximal to the transcription start site was searched using the CpG island revealing program on WebGene Home Page website (www.itba.mi.cnr.it/webgene/). Criteria for a CpG island was based on Takai and Jones: GC≧55%; Obs/Exp≧65 and length >200 bp, at cpgislands.usc.edu reported to exclude most Alu-repetitive elements (Takai et al. (2002) Proc. Natl. Acad. Sci., 99:3740-5). The RepeatMasker Web Server (ftp.genome.washington.edu/cgi-bin/RepeatMasker) was used to examine whether the promoter CpG island contained repetitive elements.

### RT-PCR

The cDNA template used for RT-PCR, was aliquoted from the same cDNA used for hybridization to the microarray. For each gene examined, primers for the housekeeping gene GAPDH were included in the RT-PCR reaction mix as a control for successful amplification. Forward and reverse primers were chosen from different exons in order to avoid amplification of any contaminating genomic DNA. RT-PCR primers are provided in Table 1.

TABLE 1
RT-PCR primers.

| | | | SEQ |
|---|---|---|---|
| | | | ID |
| Gene | Length | Sequence | NO: |
| COL1A1 | 234 bp | 5′-GATCTGCGTCTGCGACAACG-3′ (S) | 1 |
| | | 5′-CTGTCCAGGGATGCCATCTC-3′ (A) | 2 |
| IGFBP1 | 202 bp | 5′-CATCCTTTGGGACGCCATCAGTA-3′ | 3 |
| | | (S) | |
| | | 5′-CTGTCTGCTGTGATAAAATCCATTC-3′ | 4 |
| IGFBP3 | 249 bp | 5′-GTCAACGCTAGTGCCGTCAG-3′ (S) | 5 |
| | | 5′-GCTCTGAGAC TCGTAGTCAAC-3′ | 6 |
| | | (A) | |
| TGM2 | 383 bp | 5′-AGCTGGTCTTAGAGAGGTG-3′ (S) | 7 |
| | | 5′-CACAAAGCTGGATCCCTG-3′ (A) | 8 |
| GADD45A | 339 bp | 5′-AGCTCCTGCTCTTGGAGACC-3′ (S) | 9 |
| | | 5′-TGTAGTTGA ACTCACTCAGCC-3′ | 10 |
| | | (A) | |
| PLAU | | 5′-GGCAGCAATGAACTTCATCAAG-3′ | 11 |
| | | (S) | |
| | | 5′-TGGCCTTTCCTCGGTAAAAGTG-3 (A) | 12 |
| GDF15 | | 5′-GACCCTCAGAGTTGCACTC-3′ (S) | 13 |
| | | 5′-CTTGCAAGGCTGAGCTGAC-3′ (A) | 14 |
| NFKBIA | 156 bp | 5′-CGAGGAGTACGAGCAGATGGTC-3′ | 15 |
| | | (S) | |
| | | 5′-ATGGTCAGTGCCTTTTCTTCATG-3 | 16 |
| | | (A) | |
| CTGF | | 5′-CACAAGGGCCTATTCTGTC-3′ (S) | 17 |
| | | 5′-CAACCACGGTTTGGTCCTTG-3′ (A) | 18 |
| NP | 196 bp | 5′-GGAGCAGGCTCATCGAGAAGG-3′ (S) | 19 |
| | | 5′-GGGGATTTCACCGTAGTCAAAG-3′ | 20 |
| | | (A) | |
| BIRC-3 | | 5′-ACACAGTTTCTAATCTGAGCATGC-3′ | 21 |
| | | (S) | |
| | | 5′-CCAGATTCCCAACACCTGAGTC-3′ | 22 |
| | | (A) | |
| CYCS | 158 bp | 5′-CGTGTCCTTGGACTTAGAGAG-3′ (S) | 23 |
| | | 5′-CACACCGTTGAAAAGGGAGG-3′ (A) | 24 |

S: sense; A: antisense; Length: basepairs of amplified product.

### Bisulfite Modification of DNA

Genomic DNA (1 μg) from untreated cell line cultures and from normal kidney tissue and primary tumors was denatured by NaOH (0.2M) for 10 min at 37° C. and then modified by hydroquinone and sodium bisulfite treatment at 50° C. for 17 hours under a mineral oil layer. Modified DNA was purified using the Wizard DNA Clean-Up system (Promega, Madison, Wis.). Modification was completed by NaOH (0.3M) treatment for 5 minutes at room temperature, followed by precipitation with glycogen, 10M ammonium acetate and ethanol precipitation. Bisulfite modification of DNA results in the conversion of unmethylated cytosines to uracil, while methylated cytosines are resistant to modification and remain as cytosine.

### Bisulfite Sequencing of Gene Promoter CpG Islands

DNA fragments of 258-461 bp in size containing the promoter CpG island were PCR amplified from bisulfite-modified cell line DNAs and normal tissue DNAs for each gene analyzed. PCR products were run in a 1.5% agarose gel, the gel slice comprising the PCR product was purified by Qiaquick (Qiagen, Valencia, Calif.), and the PCR product was directly sequenced. Table 2 provides the primers employed. In addition, some gene products were cloned into a TOPO vector (Invitrogen, Carlsbad, Calif.) and at least ten colonies from each gene analyzed by sequencing.

TABLE 2
Bisulfite sequencing primers.

| | | | SEQ |
|---|---|---|---|
| | | | ID |
| Gene | Length | Sequence | NO: |
| COL1A1 | 461 bp | 5′-GGTAGTTTTGATTGGTTGGGGTA-3′ (S) | 25 |
| | | 5-CCCTCATCATCTCCCTTCCATT-3′ (A) | 26 |
| IGFBP1 | 357 bp | 5-YGTAGGGTTTTGGGTGTATTAGTA-3′ (S) | 27 |
| | | 5-CAATCAACAAAAACAATACCAACCAAA-3′ (A) | 28 |
| IGFBP3 | 432 bp | 5-TGTTGAGGTGGTTTGGAG-3′ (S) | 29 |
| | | 5-CAACACCAACAAAATCAAC-3′ (A) | 30 |
| IGFBP3(N) | 346 bp | 5-TGTTGAGGTGGTTTGGAGTGT-3′ (S) | 31 |
| | | 5-AACTATAAAATCCAAACAAAAAAC-3′ (A) | 32 |
| TGM2 | 399 bp | 5-GTATTTTGGGTTAGTTGTGTG-3′ (S) | 33 |
| | | 5-CAAATCAAAACTTAAAAATTCAACTC-3′ (A) | 34 |
| TGM2 (N) | 258 bp | 5-GGGTTTYGGTTTTTTGGGTGA-3′ (S) | 35 |
| | | 5-CRACTACRATAACTCTAATACT-3′ (A) | 36 |
| GADD45A | 383 bp | 5-GGTTAAGTTGTATGTAAATGA-3′ (S) | 37 |
| | | 5-ATCATAT TACAAACTACAAATC-3′ (A) | 38 |
| PLAU | | 5-GTTGTAAGATAGGGGAGGGA-3′ (S) | 39 |
| | | 5-CRATAACCAAACTCCCCAACT-3′ (A) | 40 |
| GDF15 | | 5-GATGTTTTTGGTGTTGTTGGTG-3′ (S) | 41 |
| | | 5-TCTCTAAAATTTCACTTACCTTCTAAC-3′ (A) | 42 |
| NFKBIA | | 5-GGTTTAGGTTTTTTTTTTTTTTTAGTAGA-3′ (S) | 43 |
| | | 5-AAAACCCTCCATAACCCACTCCT-3′ (A) | 44 |
| CTGF | | 5-TYGTTYGTAGTGTTAATTATGA-3′ (S) | 45 |
| | | 5-CCRAAATAACAAAATAAACCCT-3′ (A) | 46 |
| NP | | 5-GTTTAGGGTTGGGAAGAATTTTGA-3′ (S) | 47 |
| | | 5-CACTAAACTCAATACCAAACCTCT-3′ (A) | 48 |
| BIRC-3 | | 5-GAGTGGGTTTGTTAGGTTATTGA-3′ (S) | 49 |
| | | 5-CCRAAATAACAAAATAAACCCT-3′ (A) | 50 |
| CYCS | | 5-GGTYGGGAAAAGTTTYGAGAAGA-3′ (S) | 51 |
| | | 5-CRCCTCCCACAACTCTACCTCT-3′ (A) | 52 |

S: sense; A: antisense; N: Nested; Y: either C or T; R: either G or A; Length: basepairs of amplified product.

### Methylation Specific PCR

Bisulfite modified primary renal tumor DNAs were PCR amplified with primers specific for methylated versus unmethylated DNA for the IGFBP1, IGFBP3, COL1A1 and RASSF1A genes. The MSP primer sequences for IGFBP1, IGFBP3 and COL1A1 are provided in Table 3. RASSF1A primers were as previously described (Battagli et al. (2003) Cancer Res., 63:8695-9). PCR amplification of tumor DNA was performed for 31-35 cycles at 95° C. denaturing, 58-66° C. annealing and 72° C. extension with a final extension step of 5 minutes. In each set of DNAs modified and PCR amplified, a cell line with known hypermethylation from the bisulfite sequencing data was used as a positive control and normal renal tissue DNA as a negative control were included. After PCR, samples were run on a 6% non-denaturing acrylamide gel with appropriate size markers and the presence or absence of a PCR product analyzed.

TABLE 3
Methylation specific PCR primers.

| | | | SEQ |
|---|---|---|---|
| | | | ID |
| Gene | Length | Sequence | NO |
| COL1A1 (U) | 161 bp | 5′-TGGGGTTGGAGTAGGAGGTATGT-3′ | 53 |
| | | (S) | |
| | | 5′-TACATCAAAAAAACAATAACCA-3′ | 54 |
| | | (A) | |
| COL1A1 (M) | 156 bp | 5′-GGTCGGAGTAGGAGGTACGC-3′ | 55 |
| | | (S) | |
| | | 5′-CGTCAAAAAAACGATAACCG-3′ | 56 |
| | | (A) | |
| IGFBP1 (U) | 130 bp | 5′-TATTGTTATTTTATTTAGTGAGT | 57 |
| | | ATTTGTT-3′ (S) | |
| | | 5′-ATACTCTCTAAACTAATAACAACA | 58 |
| | | AACA-3′ (A) | |
| IGFBP1 (M) | 124 bp | 5′-CGTTATTTTATTTAGCGAGTATTTGT | 59 |
| | | C-3′ (S) | |
| | | 5′-CTCTCTAAACTAATAACGACGAAC | 60 |
| | | G-3′ (A) | |
| IGFBP3 (U) | 132 bp | 5′-GTGAAGTATGGGTTTTGTAGTTG-3′ | 61 |
| | | (S) | |
| | | 5′-AAACAACCCAAACACACCAACC-3′ | 62 |
| | | (A) | |
| IGFBP3 (M) | 82 bp | 5′-TGATTCGGGTTTCGGGC-3′ (S) | 63 |
| | | 5′-AACCGAAACCGAACTCG-3′ (A) | 64 |

S: sense; A: antisense; U: unmethylated; M: methylated; Length: basepairs of amplified product.

### Results

To determine the optimal 5Aza-dC dosage for robust detection of transcriptional reactivation on the microarray without excessive toxicity to the treated cells, the re-expression of 5 tumor suppressor genes, p16^{INK4a}, MLH1, MGMT, RARβ, and Timp-3, was examined at different doses of 5Aza-dC. The five TSG are well characterized as hypermethylated with associated silencing in the SW48 colorectal tumor cell line, which has a similar cell doubling time to the RCC lines (Wheeler et al. (1999) Proc. Natl. Acad. Sci., 96:10296-301; Esteller et al. (1999) Cancer Res., 59:793-97; Paz et al. (2003) Cancer Res., 63:1114-21; Herman et al. (1995) Cancer Res., 55:4525-30; Bachman et al. (1999) Cancer Res., 59:798-802). RNA was extracted from SW48 cell cultures after treatment with 1, 5, or 10 μM 5Aza-dC and TSA (500 nM). RT-PCR was then performed and the product labeled and hybridized to the expression array. Analysis of up-regulation of the 5 TSG demonstrated that treatment with 5 μM 5Aza-dC for at least two cell doubling times resulted in re-expression of the 5 TSG with minimal toxicity as assessed by comparison of cell morphology and cell death between control and treated cells. The 5Aza-dC treatment was combined with TSA because there is good evidence that the processes of methylation and deacetylation interact to silence transcription, although TSA may have less synergistic effect at the relatively higher 5 μM 5Aza-dC dose employed than when combined with lower doses of 5Aza-dC (Cameron et al. (1999) Nat. Genet., 21:103-7; Wade et al. (2001) Oncogene, 20:3166-73). Notably, with an epigenetic reactivation and expression array approach, sensitivity of signal might vary depending upon different baseline expression levels of genes and different types of array. Furthermore, different genes in the same cell line or the same gene locus in different cell lines may be more or less strongly epigenetically silenced. Interestingly, MLH1 showed the least up-regulation of the 5 TSGs examined in SW48 and a similar observation was noted for MLH1 reactivation in the RKO colorectal tumor cell line (Suzuki et al. (2002) Nat. Genet., 31:141-9).

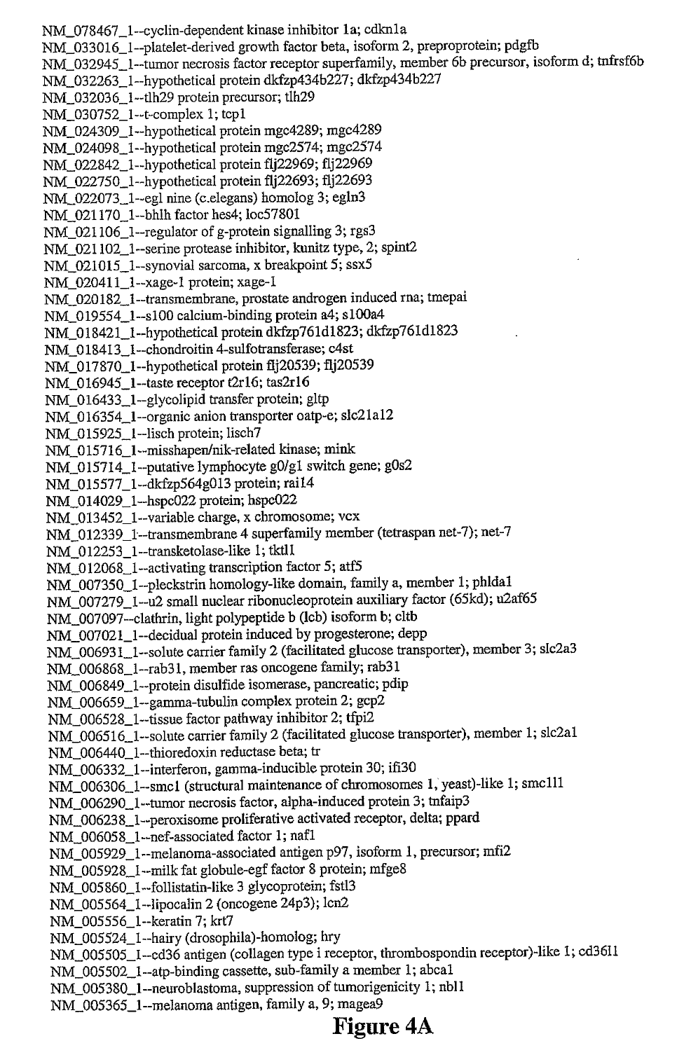

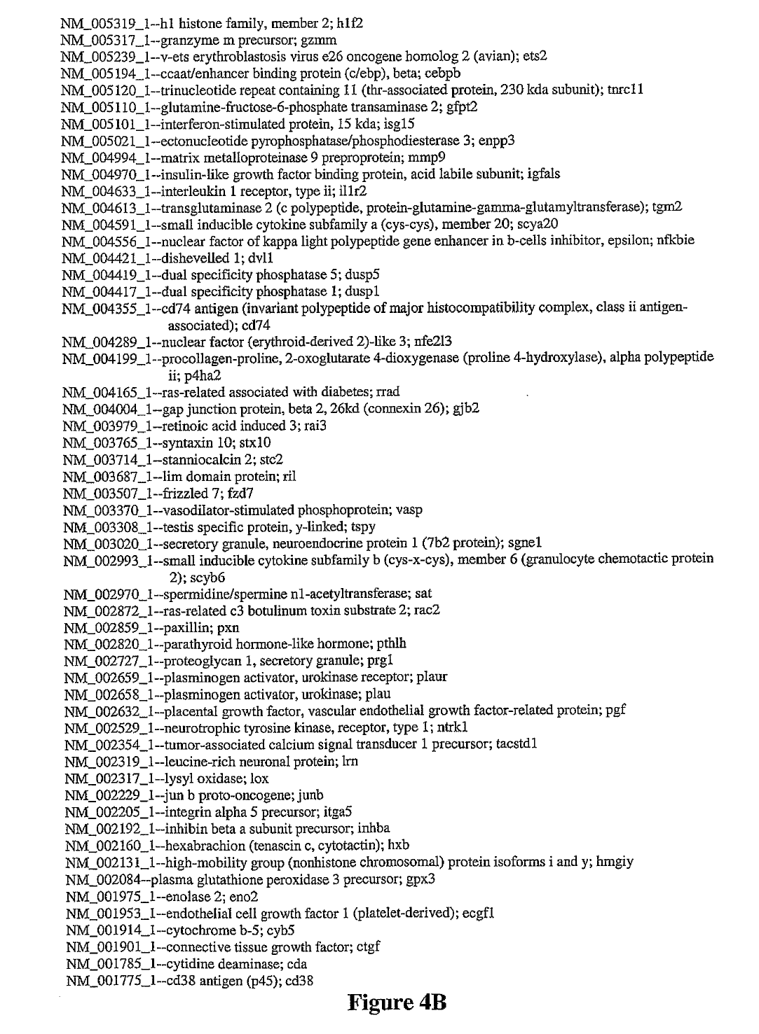

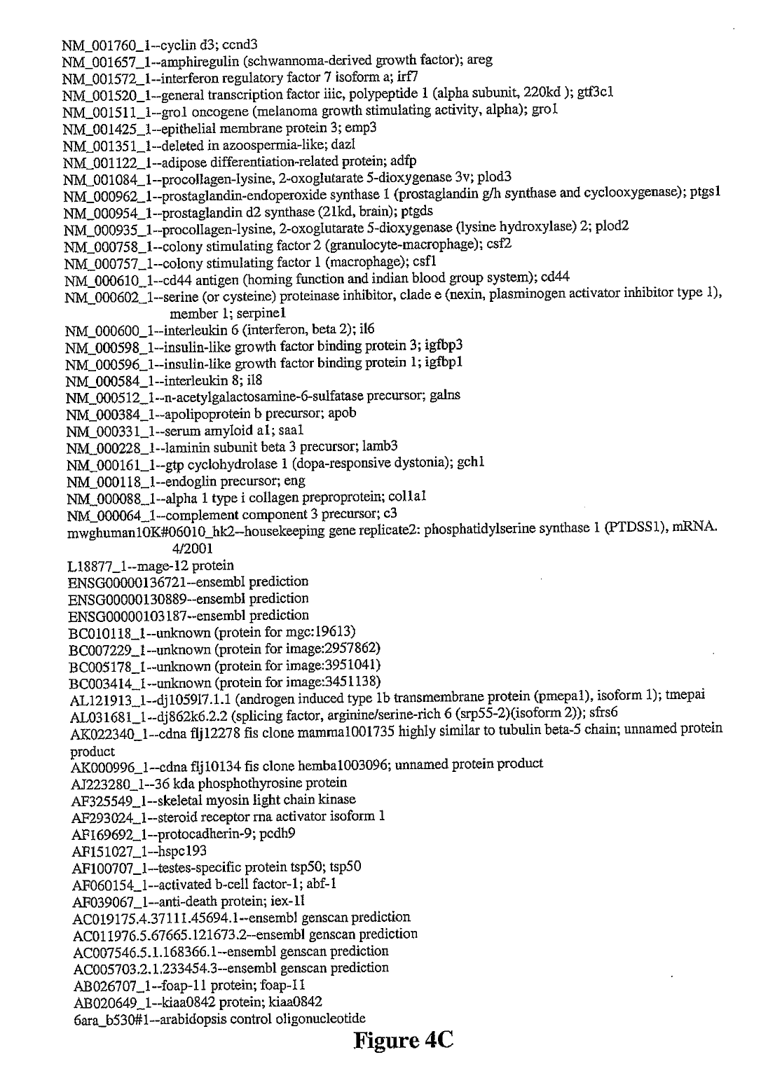

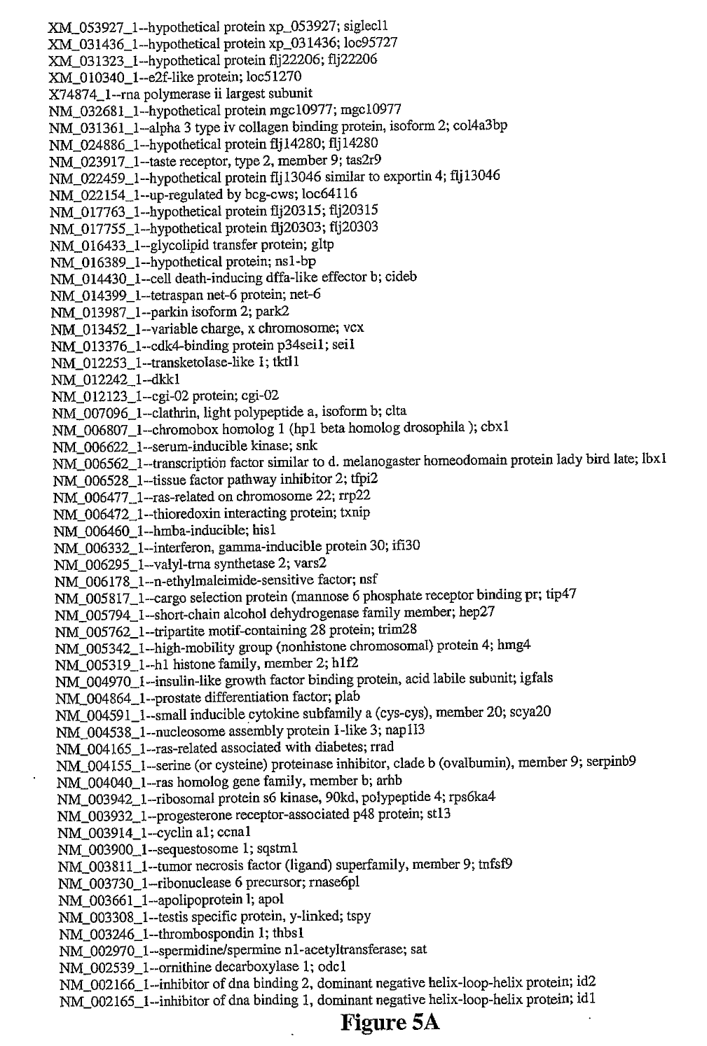

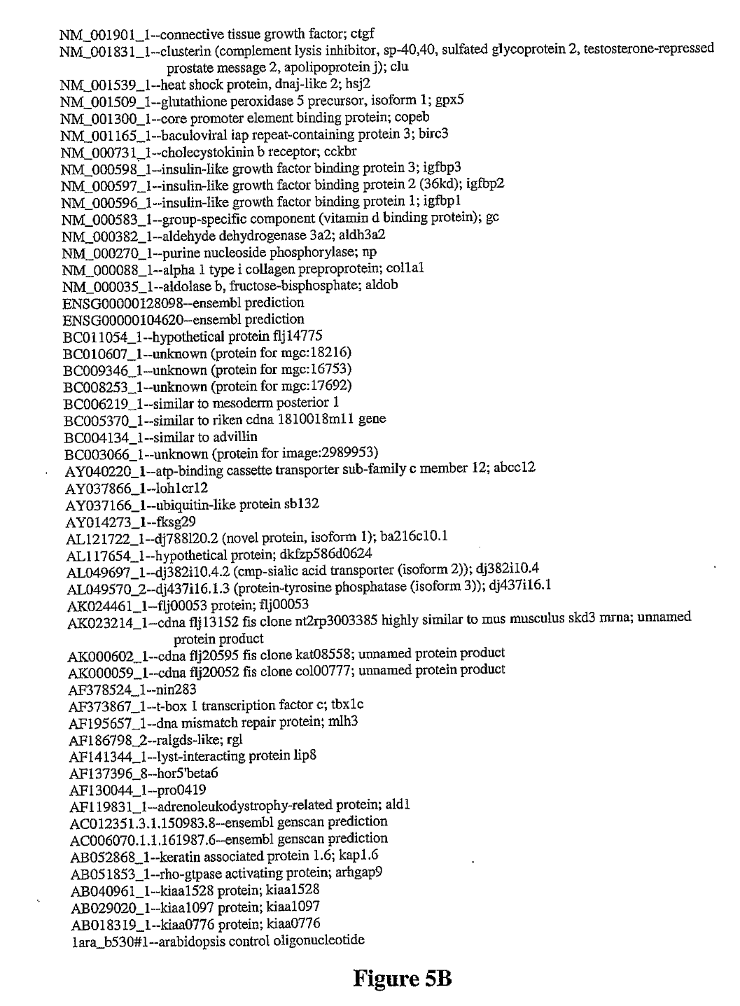

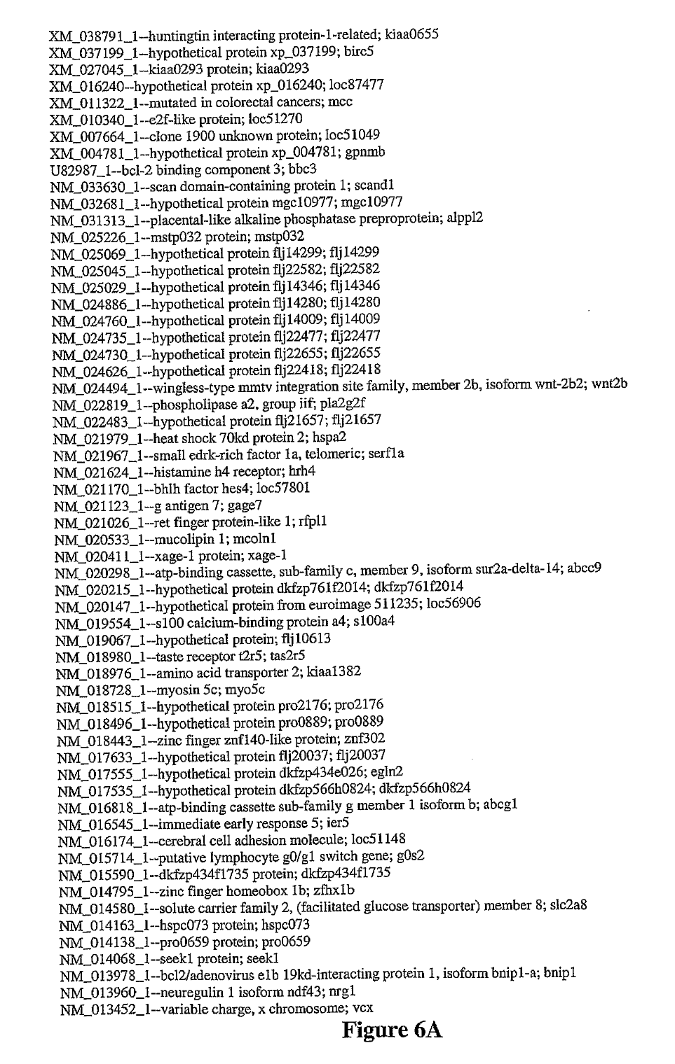

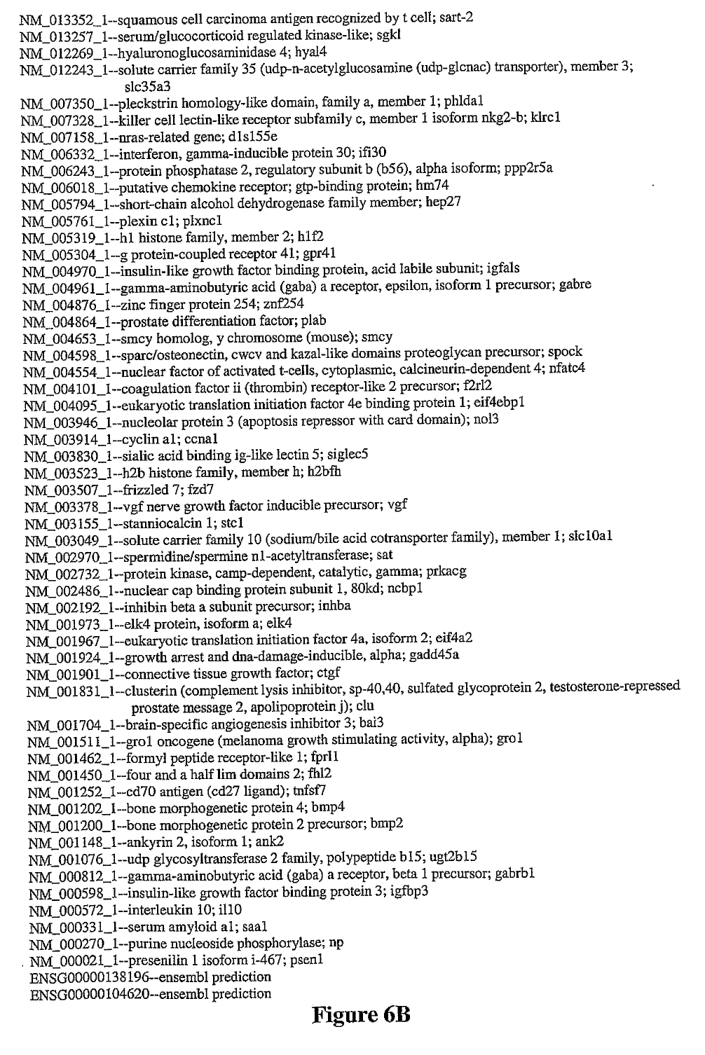

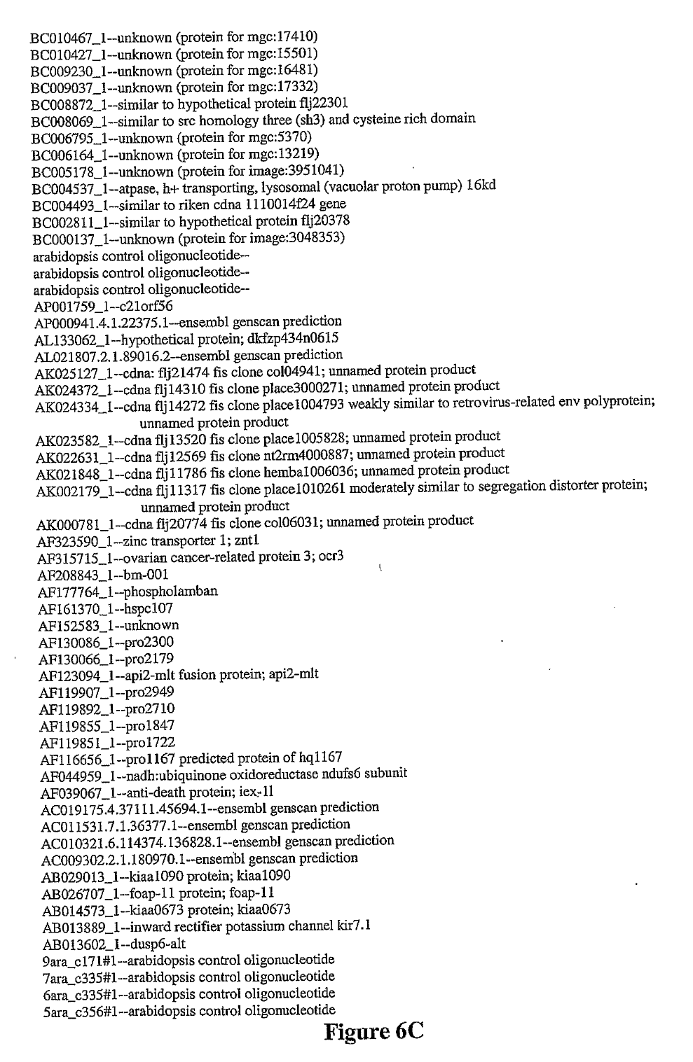

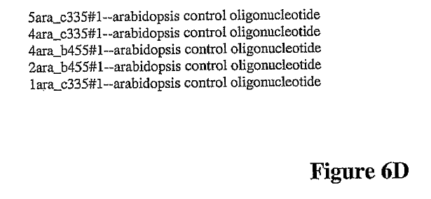

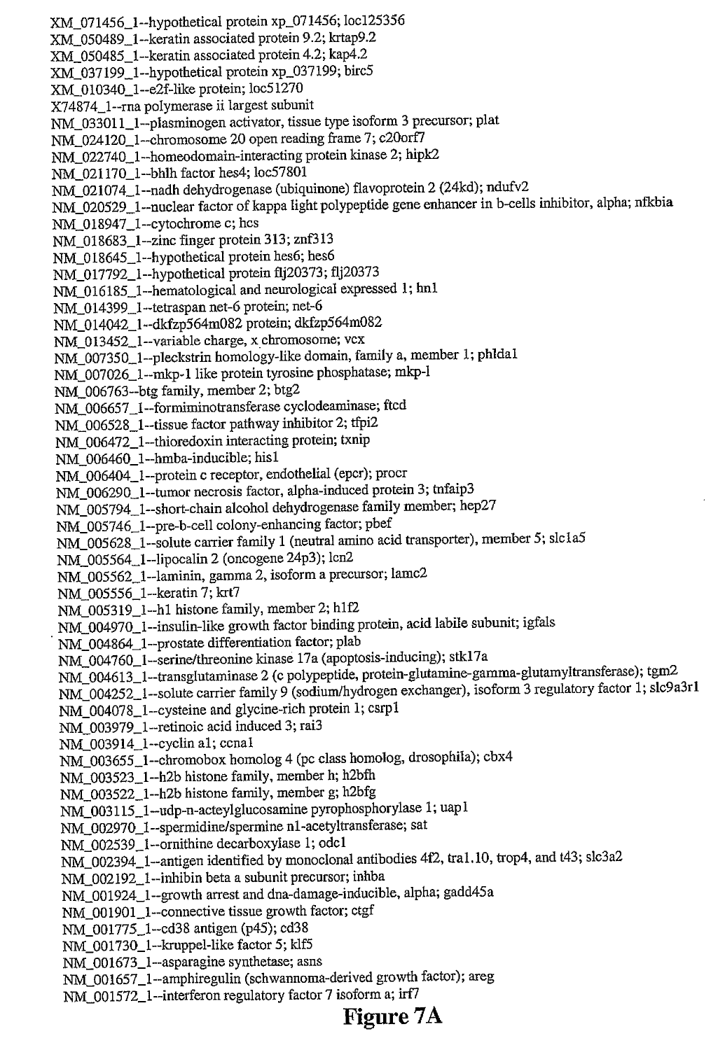

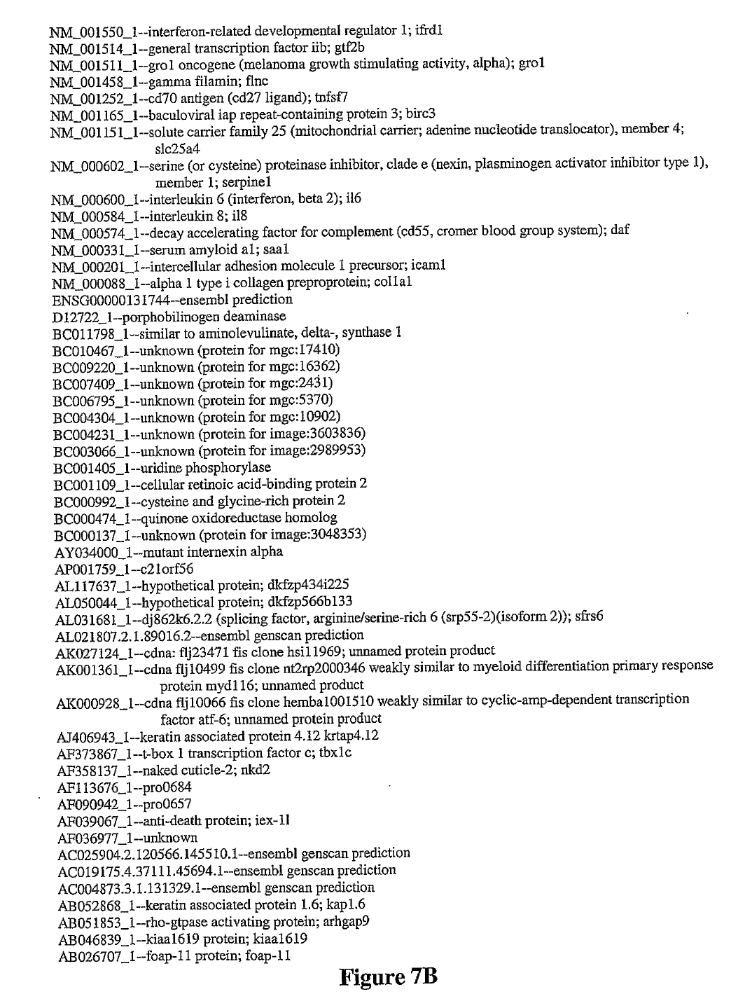

Up-regulation of the 14,802 gene microarray was analyzed in 4 RCC lines treated with 5 μM of 5Aza-dC and 500 nM of TSA. Two ATCC renal tumor cell lines (786-0 and ACHN) and two cell lines established from localized primary renal tumors were also used because the ATCC renal tumor lines were derived from clinically advanced tumors. After treatment, 170(786-0), 112 (HRC51), 178 (HRC59) and 111 (ACHN) genes were determined to be upregulated at least 3-fold in the RCC cell lines compared to the untreated cells (see FIGS. 4-7). To verify the specificity of the screen for genes hypermethylated in renal cancer, selection criteria were applied to the list of upregulated genes. 27 genes that were upregulated were identified in at least 3 of the 4 RCC lines on the basis that such genes might be expected to be frequently methylated in primary renal cancer. In addition, one gene upregulated after drug treatment in both clear cell lines but neither of the papillary lines was examined to investigate cell type specific methylation. The CGAP SAGE database was also studied to determine if each gene was known to be expressed in normal renal cells. This review lead to the exclusion of 12 genes which had no evidence of expression or no data available. The promoter region of each gene was also analyzed for the presence of a CpG island with the characteristics described by Takai and Jones (Takai et al. (2002) Proc. Natl. Acad. Sci., 99:3740-5). 14 of the 15 genes were found to have such a CpG island (Table 4) within 500 bp of either side of the transcription start site. The CpG islands of 3 genes contained repetitive elements and were excluded from the initial validation. Accordingly, the remaining 12 genes were selected for validation (Table 4).

TABLE 4
Upregulated genes identified by intuitive selection

| Gene ID | Gene Name | Gene Symbol |
|---|---|---|
| Function known | | |
| NM_001165 | Baculoviral iap repeat-containing protein 3 | BIRC3 |
| NM_001012270 | Hypothetical protein xp_037199 | BIRC5 |
| NM_024886 | Chromosome 10 open reading frame 95 | C10orf95 |
| NM_004591 | Small inducible cytokine subfamily a | CCL20 |
| NM_001831 | Clusterin | CLU |
| NM_000088 | Alpha 1 type i colagen preproprotein | COL1A1 |
| AK000928 | cAMP responsive element binding protein-like 1 | CREBL1 |
| NM_001901 | Connective tissue growth factor | CTGF |
| NM_018947 | Cytochrome c | CYCS |
| NM_001924 | growth arrest and dna-damage-inducible, alpha | GADD45A |
| NM_004864 | Prostate differentiation factor | GDF15 |
| AF039067 | Anti-death protein | IER3 |
| NM_004970 | Insulin-like growth factor binding protein, acid labile subunit | IGFALS |
| NM_000596 | Insulin like growth factor binding protein 1 | IGFBP1 |
| NM_000598 | Insulin like growth factor binding protein 3 | IGFBP3 |
| NM_000584 | Interleukin 8 | IL8 |
| NM_002192 | Inhibin beta a subunit precursor | INHBA |
| NM_020529 | Nuclear factor of kappa light polypep, gene enhan, in b-cells Inhibitor, alpha | NFKBIA |
| NM_000270 | Purine nucleoside phosphorylase | NP |
| NM_002658 | Plasminogen activator, urokinase | PLAU |
| NM_000331 | Serum amyloid a1 | SAA1 |
| NM_016521 | Transcription factor Dp family, member 3 | TFDP8 |
| NM_006528 | Tissue factor pathway inhibitor 2 | TFPI2 |
| NM_004613 | Transglutaminase 2 | TGM2 |
| NM_013452 | Variable charge | VCX3A |
| Function unknown | | |
| AC006070.1.1.161987.6 | Ensembl genscan prediction | |
| ENSG00000104620 | Ensembl prediction | |
| BC010467 | Unknown | |

| | | Chr. | | CpG | | |
|---|---|---|---|---|---|---|
| Gene ID | Function | Location | Expression | Island | ALUs | Methylation |
| Function known | | | | | | |
| NM_001165 | Apoptosis | 11q22.1 | Yes | Yes | N | U |
| NM_001012270 | Anti-apoptosis | 17q.25.3 | Yes | Yes | Y | |
| NM_024886 | Unknown | 10q24.82 | Yes | Yes | Y | |
| NM_004591 | Inflammatory response | 2q36.3 | No data | | | |
| NM_001831 | Apoptosis | 8p21.1 | Yes | No | | |
| NM_000088 | Extracellular matrix structural | 17q21.33 | Yes | Yes | N | M |
| | constituent | | | | | |
| AK000928 | transcription factor | 6p21.32 | No data | | | |
| NM_001901 | Regulation of cell growth | 6q23.2 | Yes | Yes | N | U |
| NM_018947 | Apoptosis | 7p15.3 | Yes | Yes | N | U |
| NM_001924 | DNA repair | 1p31.2 | Yes | Yes | N | U |
| NM_004864 | Growth factor activity | 19p13.11 | Yes | Yes | N | M |
| AF039067 | Apoptosis | 6p21.33 | Yes | Yes | Y | |
| NM_004970 | Insulin-like growth factor | 16p13.3 | No exp | | | |
| | binding | | | | | |
| NM_000596 | Regulation of cell growth | 7p13 | Yes | Yes | N | M |
| NM_000598 | Regulation of cell growth. | 7p14-p12 | Yes | Yes | N | M |
| | Apoptosis | | | | | |
| NM_000584 | Inflammatory response. | 4q13.3 | No exp | | | |
| NM_002192 | Tumor-suppressor. Apoptosis | 2q35 | No exp | | | |
| NM_020529 | Apoptosis | 14q13 | Yes | Yes | N | U |
| NM_000270 | DNA modification | 14q11.2 | Yes | Yes | N | U |
| NM_002658 | Signal transduction | 10q.22.2 | Yes | Yes | N | M |
| NM_000331 | Acute-phase response | 11p15.1 | No exp | | | |
| NM_016521 | Cell cycle. Transcription factor | Xq26.2 | No data | | | |
| NM_006528 | Blood coagulation | 7q21.3 | No exp | | | |
| NM_004613 | Protein signaling pathway | 20q11.23 | Yes | Yes | N | U |
| NM_013452 | Neurogenesis | Xp22.31 | No data | | | |
| Function unknown | | | | | | |
| AC006070.1.1.161987.6 | | | No data | | | |
| ENSG00000104620 | | | No data | | | |
| BC010467 | | | No data | | | |

The table shows 27 genes that showed 3-fold upregulation in at least three of the four cell lines and follow the selection criteria described hereinabove.

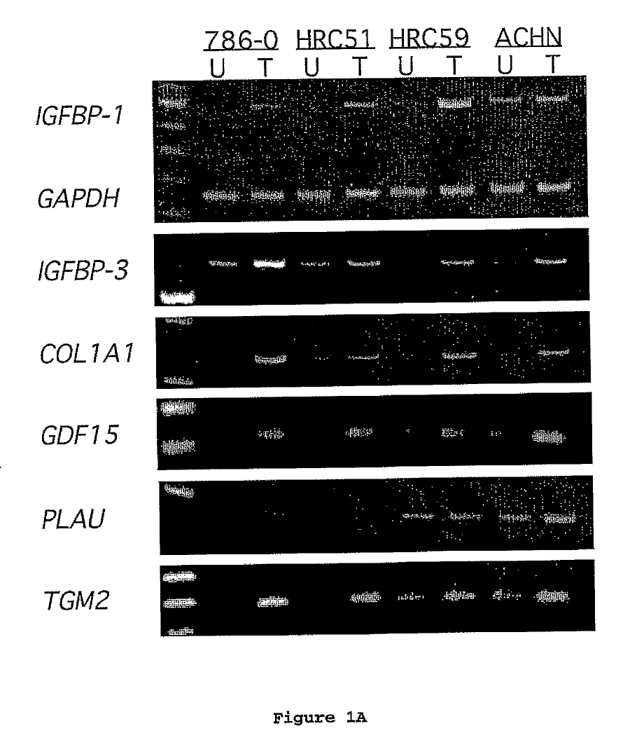

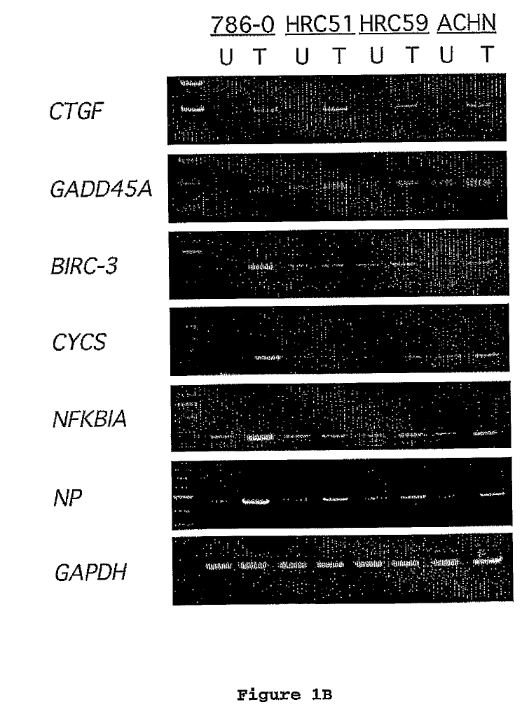

RT-PCR was performed for the 12 genes on untreated and treated cell line RNA to independently confirm the up-regulation of expression observed by array analysis (FIGS. 1A and 1B). The RT-PCR results were concordant with the microarray analysis for all 12 genes examined. For example, COL1A1 and IGFBP3 upregulation after treatment is confirmed by RT-PCR in FIG. 1A, where a strong signal from the four cell lines is seen for COL1A1 and in the HRC59 and ACHN cell lines for IGFBP3, after treatment compared with the signal obtained from the untreated cell lines. IGFBP3 also showed strong upregulation in the cell lines 786-0 and HRC51, although these two cell lines had weak basal expression of the gene before the treatment. This basal expression may represent unmethylated, or less densely methylated, sub-clones in the cell lines (Liang et al. (2002) Cancer Res., 62:961-6; Bender et al. (1999) Mol. Cell Biol., 19:6690-8).

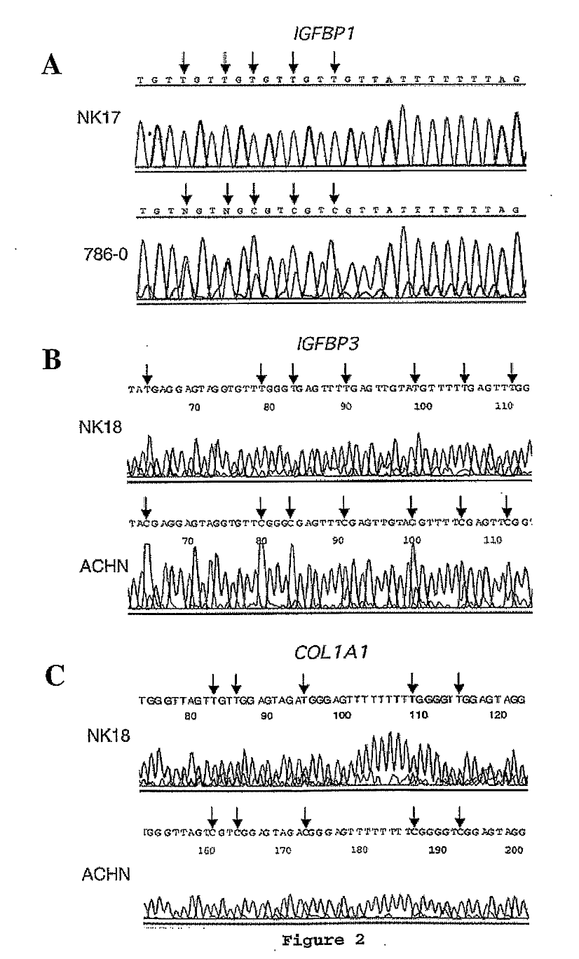

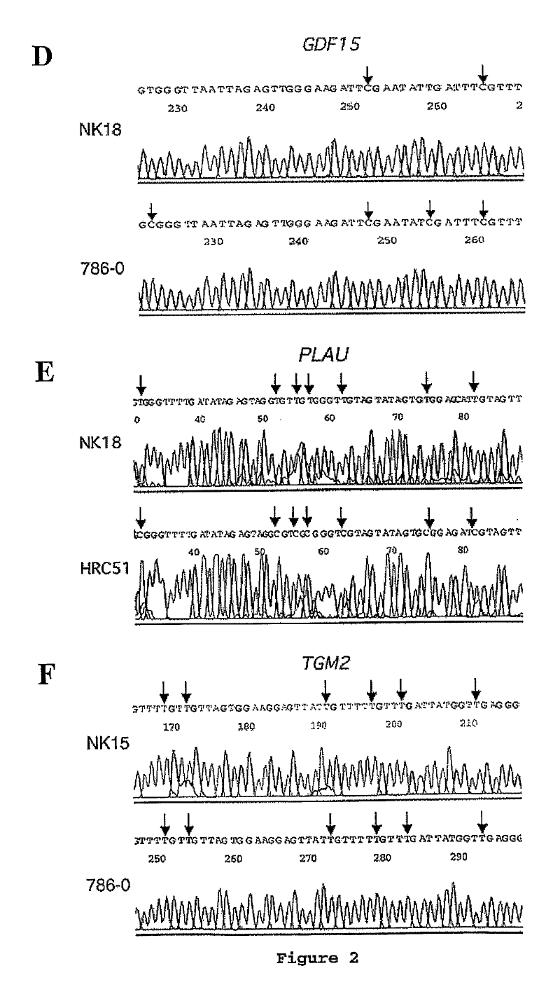

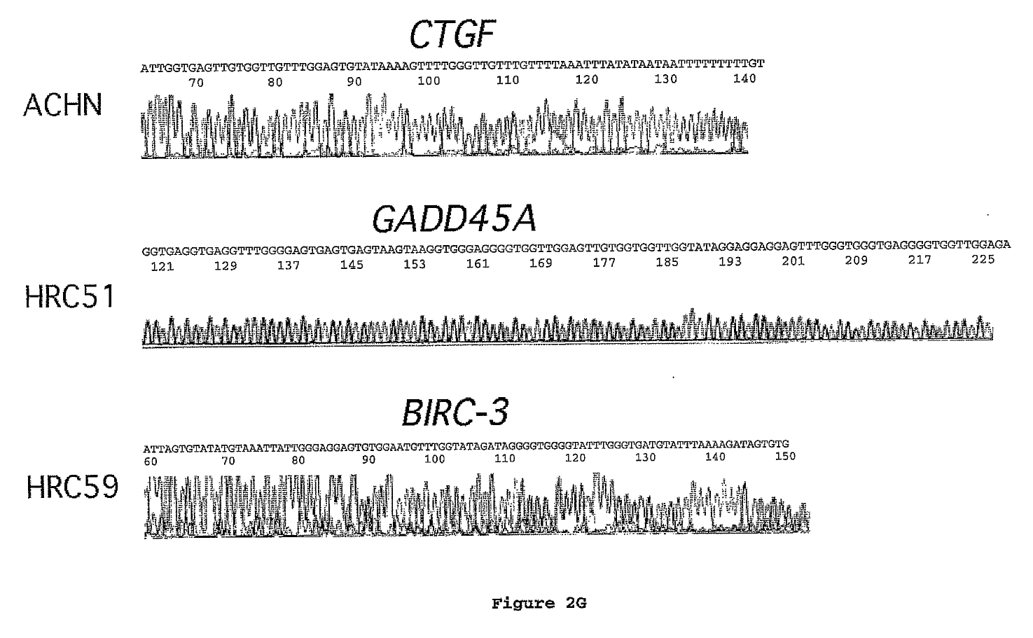

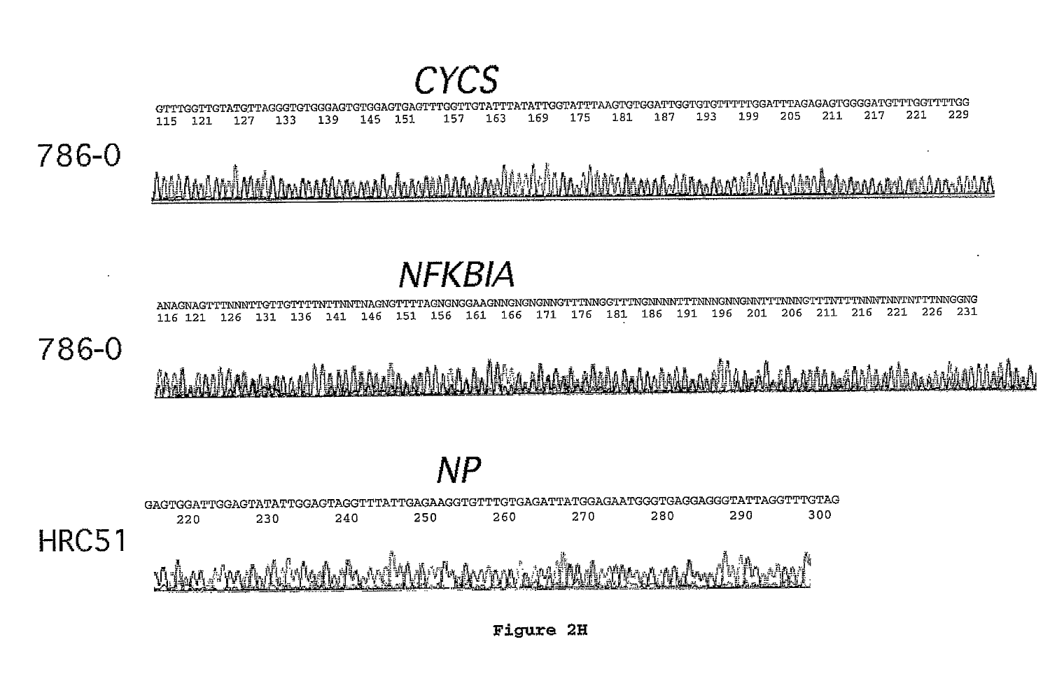

The methylation status of the promoter CpG island proximal to the transcription start site for the 12 genes was analyzed in the 4 RCC cell lines and normal renal epithelial cells by bisulfite sequencing (FIG. 2). Five genes were densely methylated in the untreated RCC lines (FIG. 2) indicating potential epigenetic regulation of these genes. Importantly, only the individual RCC lines where a particular gene was methylated showed upregulation of expression for the same gene after demethylating treatment by the microarray and RT-PCR assays, while the cell lines where a gene was unmethylated did not show upregulation after treatment. Promoter hypermethylation of IGFBP1, IGFBP3 and COL1A1 was cancer cell specific since methylated CpGs were seen only in the RCC lines and not in the normal cell DNA specimens sequenced (FIG. 2).

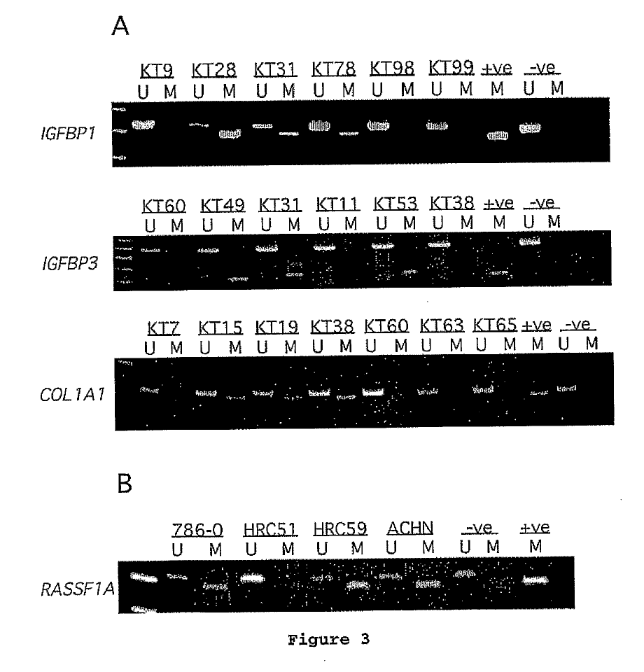

To determine if hypermethylation of the 3 genes was frequent in primary renal cancer and not limited to, or more common in, tumor cell lines, we performed MSP analysis of 32 primary organ-confined (stage I or II) renal tumors of the most common histological cell types (20 clear cell, 10 papillary and 2 chromophobe; Table 5). IGFBP1 was methylated in 10 of 32 (31%), IGFBP3 in 12 of 32 (37%), and COL1A1 in 18 of 32 (56%) primary RCC (FIG. 3A). IGFBP3 showed a similar percentage of methylation between clear cell (7/20, 35%) and papillary (4/10, 40%) tumors. IGFBP1 was more frequently methylated in clear cell (7/20, 35%) than papillary tumors (2/20, 10%) as was COL1A1 (13/20, 65% clear cell vs. 5/10, 42% papillary). Thus, the 3 genes were frequently methylated in early stage tumors of the most common histological subtypes of RCC implicating these genes in renal tumorigenesis and as novel candidate markers for the molecular detection and prognosis of kidney cancer. Further impetus has been provided by studies showing the feasibility of detecting gene hypermethylation in urine, a readily accessible bodily fluid for diagnosis and monitoring of renal cancer (Hoque et al. (2004) Cancer Res., 64:5511-7; Battagli et al. (2003) Cancer Res., 63:8695-9).

TABLE 5
Clinicopathological Data of Primary RCC.

| | Age/Sex | Cell Type | Size | Grade | Stage | IGFBP1 | IGFBP3 | COL1A1 |
|---|---|---|---|---|---|---|---|---|
| 1 | 43M | Clear Cell | 3 | I | I | U | U | M |
| 2 | 57F | Clear Cell | 3 | II | III | U | M | M |
| 3 | 59M | Clear Cell | 3.5 | III | I | U | U | M |
| 4 | 78M | Clear Cell | 2.7 | IV | I | U | U | U |
| 5 | 69M | Clear Cell | 3 | III | I | M | U | M |
| 6 | 52M | Clear Cell | 4 | IV | I | M | U | U |
| 7 | 70M | Clear Cell | 4.5 | II | I | M | U | M |
| 8 | 78M | Clear Cell | 5.5 | II-III | IV | U | U | M |
| 9 | 59M | Clear Cell | 4.5 | IV | I | M | M | M |
| 10 | 59M | Clear Cell | 9.5 | III | III | M | M | U |
| 11 | 61F | Clear Cell | 6.5 | II | I | U | U | M |
| 12 | 61M | Clear Cell | 15 | I | II | U | M | U |
| 13 | 72F | Clear Cell | 4 | II | I | U | U | U |
| 14 | 62M | Clear Cell | 6 | I-II | I | U | U | M |
| 15 | 57M | Clear Cell | 5.5 | II | I | M | M | M |
| 16 | 68M | Clear Cell | 5.5 | II | I | U | M | M |
| 17 | 60F | Clear Cell | 3.5 | II | I | U | M | M |
| 18 | 62M | Clear Cell | 3.5 | I-II | I | M | U | M |
| 19 | 59M | Clear Cell | 4 | III-IV | I | U | U | U |
| 20 | 66M | Clear Cell | 2 | I | I | U | U | U |
| 21 | 30F | Papillary | 4 | III | I | U | U | M |
| 22 | 34M | Papillary | 7.5 | II | II | U | U | U |
| 23 | 39F | Papillary | 8.5 | IV | III | M | U | M |
| 24 | 69M | Papillary | 3 | III | III | M | M | U |
| 25 | 74M | Papillary | 12.5 | II | II | U | U | U |
| 26 | 64F | Papillary | 4.5 | II | I | U | M | U |
| 27 | 57M | Papillary | 5 | III | I | U | U | U |
| 28 | 67F | Papillary | 1.2 | I | I | U | M | M |
| 29 | 73M | Papillary | 3 | I | I | U | U | M |
| 30 | 65F | Papillary | 4 | I | I | U | M | U |
| 31 | 66M | Chromophobe | 3.5 | I | I | U | M | M |
| 32 | 65F | Chromophobe | 2 | I | I | M | U | U |

Age: years;
Sex: M = Male, F = Female;
Size of tumor in cm;
Grade = American Joint Committee on Cancer;
Stage = American Joint Committee on Cancer stage grouping;
U = Unmethylated gene,
M = Methylated gene.

Aberrant hypermethylation of normally unmethylated promoter regions with associated transcriptional silencing in cancer cells is a characteristic of TSG (Baylin et al. (1998) Adv. Cancer Res., 72:141-96; Jones and Laird (1999) Nature Genet., 21:163-67). The 3 genes we have identified are therefore candidate TSG. The IGFBP1 gene maps to chromosome 7 p3, IGFBP3 to 7 p4 and COL1A1 to 17q21 respectively. These chromosomal regions have not been examined for LOH in renal cancer (Morita et al. (1991) Cancer Res., 51:820-3; Thrash-Bingham et al. (1995) Proc. Natl. Acad. Sci., 92:2854-8). High density SNP array analysis will establish if allelic deletion occurs at these loci.

In regard to the putative role of these genes in cancer, the insulin like growth factor binding proteins 1 (IGFBP1) and 3 (IGFBP3) are major forms of the IGF-binding protein family that can inhibit the growth promoting activity of both IGF I and IGF II. IGFBP-3 is known to inhibit cell growth by sequestering IGF I, however, the mechanism by which IGFBP-1 exerts its activity is less well understood. Down-regulation of IGFBP-3 expression has been reported in non-small cell lung cancer, prostate cancer, and hepatocellular carcinoma where IGFBP3 promoter hypermethylation was also described (Chang et al. (2002) Clin. Cancer Res., 8:3796-802; Chan et al. (1998) Science, 279:563-6; Gong et al. (2000) Mol. Cell Biochem., 207:101-4; Hanafusa et al. (2002) Cancer Lett., 176:149-58). IGFBP3 was also recently identified as hypermethylated in a mouse skin multistage carcinogenesis model (Fraga et al. (2004) Cancer Res., 64:5527-34). Clearly, methylation-based silencing of IGFBP1 and IGFBP3 could provide growth advantages to the neoplastic cell. Activation of this pathway may be of therapeutic advantage in limiting tumor growth.

COL1A1 is the human gene coding for the at chain of type I collagen, the major extracellular matrix component of skin and bone. Changes in the synthesis of type I collagen are associated with normal growth and tissue repair processes. The related genes COL1A2 and COL1A5 have also been reported to be hypermethylated in cancer cells (Sengupta et al. (2003) Cancer Res., 63:1789-97; Paz et al. (2003) Hum. Mol. Genet., 12:2209-19). Alterations in extracellular matrix composition have been implicated in tumor progression and metastasis. Both the IGFBP and the COL1A gene families appear prone to hypermethylation and it is interesting that other global epigenetic screens have shown reactivation of gene families e.g. IFN in bladder and SFRP family members in colorectal cancer (Liang et al. (2002) Cancer Res., 62:961-6; Suzuki et al. (2002) Nat. Genet., 31:141-9).

The fourth reactivated gene found to have promoter methylation was a growth differentiation factor gene, GDF15, located on chromosome 19p. GDF15 showed methylation of CpG dinucleotides in normal cell DNA, but denser CG methylation in the tumor cell DNA. Hypermethylation of GDF15 in normal cells may be age-related as described for myoD1 and other cancer genes (Ahuja et al. (1998) Cancer Res., 58:5489-94). The fifth gene, PLAU, was densely methylated in HRC51 but this line did not show significant upregulation after treatment by RT-PCR. The other 3 lines did show PLAU upregulation by RT-PCR but all had unmethylated promoters.

The remaining 7 upregulated genes had unmethylated promoters by bisulfite sequencing analysis (FIG. 2) but could have been activated by an upstream regulatory gene or transcription factor that was reactivated by demethylation. In support of this idea, TGM2 has been reported to be regulated by the candidate TSG, RASSF1A, known to be frequently methylated in RCC (Agathanggelou et al. (2003) Cancer Res., 63:5344-51; Morrissey et al. (2001) Cancer Res., 61:7277-81; Yoon et al. Int. J. Cancer, 94:212-7; Dulaimi et al. (2004) Clin. Cancer Re., 10(12 Pt 1):3972-9). RT-PCR confirmed clear upregulation of TGM2 in the 4 RCC lines after treatment (FIG. 1) and, as such, can be a cancer marker. RASSF1A was examined by MSP and found to be methylated in 3 of the 4 RCC lines (FIG. 3B). It is also likely that the epigenetic reactivation of particular genes leads to a cascade of upregulation in diverse pathways and networks. Other genes may be upregulated as a direct response to the stress of 5Aza-dC treatment.

While the invention has been described in detail and with reference to specific examples thereof, it will be apparent to one skilled in the art that various changes and modifications can be made therein without departing from the spirit and scope thereof.

What is claimed is:

1. A method for the detection of cancer, comprising,
a) providing a biological sample obtained from a patient;
b) determining the methylation pattern of at least one nucleic acid molecule of said biological sample; and
c) comparing the methylation pattern of said nucleic acid molecule from said patient with the methylation pattern of said nucleic acid molecules from a normal subject,
wherein hypermethylation of said nucleic acid molecule obtained from said patient relative to the methylation pattern from said normal subject is indicative of the presence of cancer, and
wherein said nucleic acid molecule comprises the promoter region of at least one gene selected from the group consisting of IGFBP1, IGFBP3, COL1A1, GDF15, and PLAU.

2. The method of claim 1, wherein said determination of said methylation pattern is achieved by performing methylation specific polymerase chain reaction on the nucleic acid molecules of said biological sample.

3. The method of claim 1, wherein said cancer is selected from the group consisting of renal, ovarian, bladder, and colorectal cancer.

4. The method of claim 3, wherein said cancer is renal cancer.

5. The method of claim 1, wherein said nucleic acid molecules comprises the promoter region of at least one gene selected from the group consisting of IGFBP1, IGFBP3, and COL1A1.

6. The method of claim 5, wherein said nucleic acid molecules comprise the promoter region of IGFBP1.

7. The method of claim 4, wherein said biological sample is selected from the group consisting of urine, kidney tissue, and tumor tissue.

8. The method of claim 4, wherein said patient has a kidney confined tumor.

9. The method of claim 2, wherein said nucleic acid molecules of said biological sample are isolated prior to performing said methylation specific polymerase chain reaction.

10. The method of claim 2, wherein said methylation specific polymerase chain reaction comprises treating said nucleic acid molecules with sodium bisulfite prior to amplification.

11. The method of claim 2, further comprising performing methylation specific polymerase chain reaction on the nucleic acid molecules of a biological sample obtained from a normal subject.

12. A kit for practicing the method of claim 2, comprising
a) primers specific for performing methylation specific PCR of the promoter region of at least one of the genes selected from the group consisting of IGFBP1, IGFBP3, COL1A1, GDF15, and PLAU; and
b) at least one hypermethylated nucleic acid molecule for use as a positive control.

13. The kit of claim 12 further comprising at least one component selected from the group consisting of:
a) at least one unmethylated nucleic acid molecule for use as a negative control;
b) reagents suitable for performing methylation specific PCR;
c) reagents suitable for performing non-denaturing gel electrophoresis; and
d) instruction material.

14. The kit of claim 12, wherein the primers are specific for the promoter region of IGFBP1.
